(19) United States
(12) Patent Application Publication
(10) Pub. No.: US 2025/0387321 A1
(43) Pub. Date: Dec. 25, 2025

(54) METHODS OF DISPERSING ARIPIPRAZOLE INJECTABLE PREPARATIONS

(71) Applicant: Otsuka Pharmaceutical Co., Ltd., Tokyo (JP)

(72) Inventors: Kazuya NAKAMURA, Osaka-shi, Osaka (JP); Yoko SAKAMOTO, Osaka-shi, Osaka (JP); Daiki KANEKO, Osaka-shi, Osaka (JP)

(21) Appl. No.: 18/249,975

(22) PCT Filed: Mar. 17, 2023
(86) PCT No.: PCT/JP2023/010713

§ 371 Date: Apr. 21, 2023

(51) Int. Cl. A61K 9/00; A61K 9/10; A61K 31/496; A61K 47/02; A61K 47/10; A61K 47/38

(52) U.S. Cl. CPC A61K 9/0019; A61K 9/10; A61K 31/496; A61K 47/02; A61K 47/10; A61K 47/38

(57) ABSTRACT

The present disclosure is directed to methods of dispersing injectable preparations comprising aripiprazole or a salt thereof. Methods of enhancing slidability of long-acting injectable preparations comprising aripiprazole or a salt thereof, comprising dispersing ingredients comprised in the preparations before administration, aripiprazole injectable preparations to be administered after the dispersion, methods of administration thereof, and use thereof are provided.

### TECHNICAL FIELD

The present disclosure is directed to methods of dispersing injectable preparations comprising aripiprazole or a salt thereof. The present disclosure is also directed to aripiprazole injectable preparations administered after dispersion, methods of administration and use thereof.

### BACKGROUND ART

Injectable preparations comprising aripiprazole or a salt thereof, also herein referred to as “aripiprazole injectable preparations” or “injectable preparations”, become a gel upon standing, which precipitation and caking of the particles of aripiprazole can be inhibited, thereby providing excellent storage stability. Furthermore, the injectable preparations, even in the form of a gel, can easily increase fluidity, which means that the preparations become a sol, when subjected to a mild impact, and the preparation can be easily injected at the time of use (PTL 1).

### CITATION LIST

### Patent Literature

[PTL 1] WO 2021/201238A1 pamphlet
[PTL 2] U.S. Pat. No. 10,517,951B2

### SUMMARY OF INVENTION

### Technical Problem

An aripiprazole injectable preparation is provided in the form of a prefilled syringe as a ready to use (RTU) preparation (PTL 2). Upon standing or static conditions, ho-mogencity of the ingredients comprised in the RTU preparation diminished and a part of the ingredients became deposited depending on the conditions during transportation or storage after formulation, resulting in the decrease of the slidability of the preparation upon use or injection and the increase of resistance upon injection.

### Solution to Problem

The present inventor has found the above problem, and after extensive studies, noted that the problem can be resolved by conducting a certain dispersion procedure to the aripiprazole injectable preparation before use, resulting in the completion of the invention.

In one aspect, a method of enhancing slidability of a container of an aripiprazole, or salt thereof, long-acting injectable preparation comprising:
dispersing a gel of the preparation to form a sol or a suspension of the preparation in the container before administration,
wherein long-acting is about two months is provided.

In another aspect, a long-acting injectable preparation for intramuscular administration for treating a subject having schizophrenia or bipolar I disorder, comprising aripiprazole or a salt thereof, in a container, wherein the preparation is in a form of a gel upon static conditions and reverts to a sol or a suspension when dispersed and long-acting is about two months is provided.

In still another aspect, a method for treating a subject having schizophrenia or bipolar I disorder, comprising dispersing an injectable preparation in a form of a gel to a sol or a suspension of aripiprazole or a salt thereof in a container, and intramuscularly administering to a subject the injectable preparation, wherein the subject is administered the injectable preparation about once every two months is provided.

In still another aspect, use of aripiprazole or a salt thereof in the manufacture of a medicament for treating a subject having schizophrenia or bipolar I disorder, wherein the medicament is a long-acting injectable preparation for intramuscular administration comprising aripiprazole or a salt thereof in a container, the preparation is in a form of a gel upon static conditions and reverts to a sol or a suspension when dispersed, and long-acting is about two months is provided.

### Advantageous Effects of Invention

Even if homogeneity of the ingredients comprised in the aripiprazole injectable preparation became diminished due to transportation and conveyance, etc., dispersion procedures to the preparation before use (or administration) may allow for redispersion of the ingredients and enhance slidability of the ingredients upon use or injection.

### BRIEF DESCRIPTION OF DRAWINGS

FIG. 1 shows slidability signals of a sample before vibration.

FIG. 2 shows slidability signals of a sample after 0 times of tapping and 0 seconds of shaking after vibration.

FIG. 3 shows slidability signals of a sample after 5 times of tapping and 5 seconds of shaking after vibration.

FIG. 4 shows slidability signals of a sample after 10 times of tapping and 10 seconds of shaking after vibration.

FIG. 5 shows slidability signals of a sample after 15 times of tapping and 15 seconds of shaking after vibration.

FIG. 6 shows slidability signals of a sample after 5 times of tapping and 0 seconds of shaking after vibration.

FIG. 7 shows slidability signals of a sample after 10 times of tapping and 0 seconds of shaking after vibration.

FIG. 8 shows slidability signals of a sample after 15 times of tapping and 0 seconds of shaking after vibration.

FIG. 9 shows slidability signals of a sample after 0 times of tapping and 5 seconds of shaking after vibration.

FIG. 10 shows slidability signals of a sample after 0 times of tapping and 10 seconds of shaking after vibration.

FIG. 11 shows slidability signals of a sample after 0 times of tapping and 15 seconds of shaking after vibration.

### DESCRIPTION OF EMBODIMENTS

As used herein, “a” or “an” entity refers to one or more of that entity, e.g., “a compound” refers to one or more compounds or at least one compound unless stated otherwise. As such, the terms “a” (or “an”), “one or more”, and “at least one” are used interchangeably herein.

As used herein, the term “about” means approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 5%.

As used herein, the term “treat,” “treating,” or “treatment,” when used in connection with a disorder or condition, includes any effect, e.g., lessening, reducing, modulating, ameliorating, or eliminating, that results in the improvement of the disorder or condition. Improvements in or lessening the severity of any symptom of the disorder or condition can be readily assessed according to standard methods and techniques known in the art. In some embodiments, the presently disclosed methods can be used to treat schizophrenia and bipolar I disorder, as maintenance monotherapy. In further embodiments, the presently disclosed methods can be used to treat schizophrenia, acute treatment of manic and mixed episodes associated with Bipolar I disorder, major de-pressive disorder (MDD), irritability with Autistic Disorder, and Tourette's disorder.

As used herein, reference to “aripiprazole” is to aripiprazole or a salt thereof, the crystalline form of aripiprazole or a salt thereof. Aripiprazole or a salt thereof may be in a monohydrate form (aripiprazole hydrate A) or in various anhydrous forms, which are known to exist in the form of anhydrous crystal B, anhydrous crystal C, anhydrous crystal D, anhydrous crystal E, anhydrous crystal F, and anhydrous crystal G. All of these crystalline forms may be used as aripiprazole or a salt thereof in the injectable preparation of the present disclosure and further for example, aripiprazole is a monohydrate form. As used herein, the term “aripiprazole” or salt thereof refers to a compound having the structure:

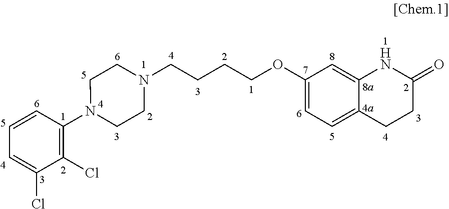 [SMILES: O=C1CCc2ccc(OCCCCN3CCN(c4cccc(Cl)c4Cl)CC3)cc2N1]

### Dispersion Procedure

The term “dispersion procedure” or “dispersing step” in the present disclosure refers to a procedure to redisperse ingredients comprised in the injectable preparation of the present disclosure before administration so as to enhance homogeneity, slidability, and/or fluidity of the preparation compared to those before the dispersion procedure, but is not limited thereto. The dispersion procedure comprises, for example, a procedure that makes the force to eject or discharge the preparation from a container, e.g., a syringe or prefilled syringe, 30N or less, such as, 25N or less, as sliding resistance. In some aspects, the dispersion procedure is a procedure conducted manually by an adult such as a patient and a healthcare practitioner or with a medical device commonly used in the art, comprising tapping the preparation at least 6 times and shaking the preparation for 6 seconds until the preparation becomes homogeneous, such as tapping at least 8 times and shaking for at least 8 seconds, further for example tapping at least 10 times and shaking for at least 10 seconds, and still further for example tapping 10 to 15 times and shaking for 10 to 15 seconds. The term “tapping” in the present disclosure refers to an act or treatment to impart motion or vibration or have an impact to the preparation. The term “shaking” in the present disclosure refers to an act or treatment to mix the preparation to make homogeneous. In some aspects, the shaking in the dispersion procedure is conducted after the tapping until the preparation becomes homogeneous. In some aspects, the dispersion procedure is conducted at ambient temperatures, such as 15° C. to 30° C. In some aspects, the preparation to which a syringe needle suitable for a subject or a patient is attached after the dispersion procedure is administered intramuscularly. The dispersion procedure of the present disclosure may be optionally adjusted depending on the content and/or shape of a container comprising the injectable preparation of the present disclosure, including a prefilled syringe. The decrease of homogeneity, slidability, or fluidity of ingredients comprised in the injectable preparation of the present disclosure may be caused by intermittent or continuous mild vibration or impact due to the conditions during transportation or storage after formulation of the preparation, particularly at low temperatures, such as 1° C. to 15° C. or below. When the homogeneity, slidability, or fluidity is decreased, the force to eject the preparation from a container comprising the preparation, also herein referred to as “sliding resistance”, may exceed 30N to eject the content in excess of a certain amount of the content unless the dispersion procedure of the present disclosure is conducted. Because even the gelled injectable preparation of the present disclosure can readily increase fluidity by simply pressing a plunger of a syringe and ejecting the preparation through a syringe needle to form a sol, in some aspects, the preparation may be used as is after an interval without vibration or impact due to the conditions during transportation or storage after the dispersion procedure of the present disclosure is conducted once for the preparation.

### Injectable Preparation

An injectable preparation of the present disclosure includes two month ready to use (RTU) injectable preparation that is disclosed in PTL 1, for example, and is a composition comprising aripiprazole, a specific suspending agent (suspending agent (A)), and a dispersion medium. For example, the injectable preparation of the present disclosure comprises at least water as a dispersion medium. Water, or an aqueous solvent comprising water and an organic solvent can be used as a dispersion medium comprising at least water. In some aspects, the dispersion medium is water, and further for example, sterile water for injection.

The specific suspending agent (suspending agent A) contained in the injectable preparation of the present disclosure comprises at least one suspending agent chosen from (i) and (ii): (i) polyvinylpyrrolidonc, and (ii) polyethylene glycol and carboxymethyl cellulose or a salt thereof.

In some aspects, the injectable preparation of the present disclosure comprises aripiprazole or a salt thereof, water, and at least one suspending agent chosen from (i) and (ii): (i) polyvinylpyrrolidone, and (ii) polyethylene glycol and carboxymethylcellulose or a salt thereof, wherein aripiprazole or a salt thereof has a mean primary particle diameter ranging from about 0.5 μm to about 30 μm and the concentration of aripiprazole or a salt thereof ranges from about 200 mg/mL to about 400 mg/mL. The presently disclosed, two month ready to use (RTU) injectable preparation is described in PTL 2, which is incorporated herein by reference in its entirety.

The injectable preparation of the present disclosure comprises aripiprazole or a salt thereof, a combination of the use of a specific suspending agent (suspending agent A) and a specific concentration of aripiprazole or a salt thereof (from about 200 mg/mL to about 400 mg/mL such as from about 250 mg/mL to about 350 mg/mL) is used. When the injectable preparation of the present disclosure comprises a salt of aripiprazole, the concentration described above is calculated as aripiprazole (in anhydrate terms).

In some aspects, in the aripiprazole injectable preparation of the present disclosure, when polyvinylpyrrolidone is contained as suspending agent A, the concentration of polyvinylpyrrolidone ranges from about 0.1 mg/mL to about 100 mg/mL, such as about 1 mg/mL to about 50 mg/mL, and further for example about 2 mg/mL to about 20 mg/mL.

In some aspects, when the aripiprazole injectable preparation of the present disclosure comprises polyethylene glycol and carboxymethylcellulose or a salt thereof as suspending agent A, the concentration of polyethylene glycol is about 0.05 mg/ml to about 2 mg/mL and further for example, about 0.1 mg/mL to about 1 mg/mL. The concentration of carboxymethylcellulose or a salt thereof is about 0.5 mg/mL to about 50 mg/mL, such as about 1 mg/mL to about 30 mg/mL, and such as about 2 mg/mL to about 20 mg/mL.

By containing carboxymethylcellulose or a salt thereof, an increase in viscosity during production can be suppressed. This allows aripiprazole or a salt thereof to be pulverized into a desirable particle size in an efficient manner. In some embodiments, the molecular weight of carboxymethylcellulose or a salt thereof ranges from 49,000 to 300,000. Furthermore, by containing polyethylene glycol, syneresis can be prevented even when the resulting injectable preparation is stored for a long period of time. In some embodiments, the molecular weight of polyethylene glycol ranges from 400 to 4,000.

In some aspects, in the aripiprazole injectable preparation of the present disclosure, when several suspending agents are used, the composition comprises about 0.5 mg/mL to about 20 mg/mL of polyvinylpyrrolidone, about 0.1 mg/mL to about 100 mg/ml of polyethylene glycol, about 0.5 mg/mL to about 50 mg/mL of carboxymethylcellulose or a salt thereof, and about 250 mg/mL to about 450 mg/mL (such as about 300 mg/mL to about 400 mg/mL) of aripiprazole or a salt thereof. In this case, the polyethylene glycol may be polyethylene glycol 400 or polyethylene glycol 4000. In some aspects, the polyvinylpyrrolidone has a K value of about 12 to about 20. In further aspects, the aripiprazole or a salt thereof has a mean primary particle diameter of about 1 μm to 10 μm.

Because an unduly large mean primary particle diameter of the aripiprazole or a salt thereof may cause precipitation, the mean primary particle diameter ranges from about 0.5 μm to about 30 μm and for example, about 1 μm to about 20 μm. When the injectable preparation of the present disclosure is in a dosage form that is administered once every two months, the aripiprazole or a salt thereof has a mean primary particle diameter of about 1 μm to about 50 μm, such as about 1 μm to about 10 μm, and further for example, about 2 μm to about 5 μm. The mean secondary particle diameter is up to but not exceeding three times and further for example, up to but not exceeding twice the mean primary particle diameter.

In some aspects, the injectable preparation of the present disclosure is suitably formulated into a dosage form that can be administered once every two months (also herein referred to as “two-month formulation” or “two-month preparation”). For example, the concentration of aripiprazole or a salt thereof in the injectable preparation of the present disclosure that is administered once every two months is calculated as aripiprazole, about 200 mg/mL to about 400 mg/mL, such as about 250 mg/mL to about 350 mg/mL, and further for example, about 300 mg/mL. When the injectable preparation is administered once every two months, the dosage volume is about 2 mL to about 4 mL, such as about 2.2 mL to about 3.5 mL, further for example, about 2.3 mL to about 3.4 mL, and further for example, about 3.2 mL.

In some aspects, the injectable preparation of the present disclosure is a long-acting injectable preparation comprising aripiprazole or a salt thereof and a specific suspending agent, which is in a sol-gel form. And the preparation may be in the form of a gel when stored and allowed to stand, and may change to a sol when subjected to an impact. In some aspects, the injectable preparation of the present disclosure may be formulated into a dosage form of a prefilled sol-gel formed injection (also herein referred to as a “prefilled sol-gel formed injectable preparation”). In some aspects, the injectable preparation of the present disclosure exhibits a thixotropic property.

In some aspects, a method for producing the aripiprazole injectable preparation is described in PTL 2, and for example, comprises preparing a liquid mixture of the starting materials and pulverizing aripiprazole or a salt thereof contained in the liquid mixture to a desired mean primary particle diameter, optionally followed by aging.

In some aspects, a method for producing a gel injectable preparation of the present disclosure comprises allowing a liquid mixture to stand at 5° C. to 70° C. for 5 minutes or more, the liquid mixture comprising aripiprazole or a salt thereof with a mean primary particle diameter of about 0.5 μm to about 30 μm in a concentration of about 200 mg/mL to about 400 mg/mL, water, and at least one suspending agent chosen from (i) and (ii): (i) polyvinylpyrrolidone and (ii) polyethylene glycol and carboxymethyl cellulose or a salt thereof.

For example, in some further aspects, a production method comprising the following steps can be used: pulverizing aripiprazole or a salt thereof to a mean primary particle diameter of about 0.5 μm to about 30 μm in a liquid mixture comprising the aripiprazole or a salt thereof in a concentration of about 200 mg/mL to about 400 mg/mL, water, and at least one suspending agent chosen from (i) and (ii): (i) polyvinylpyrrolidone and (ii) polyethylene glycol and carboxymethyl cellulose or a salt thereof; and allowing the pulverized liquid mixture to stand at 5° C. to 70° C. for about 5 minutes or more, about 10 minutes or more, about 15 minutes or more, or about 30 minutes or more and within about 1 hour or within about 24 hours.

In some aspects, the present disclosure comprises the administration of the aripiprazole injectable preparation, as two-month formulation, resulting in a total amount of aripiprazole ranging from about 700 mg to about 1000 mg, for example from about 720 mg to about 960 mg, further for example about 720 mg or about 960 mg, administered to the patient, wherein the injectable preparation may be optionally administered in one or more divided injections. In some further aspects, the administration of the aripiprazole injectable preparation results in a total amount of aripiprazole of about 960 mg to the patient, wherein one or more injectable preparations are administered. For example, to obtain a total amount of 960 mg of aripiprazole administered to the patient, one (1) injection of the aripiprazole injectable preparation comprising 300 mg/mL of aripiprazole is administered. The 960 mg dosage may be optionally administered in separate injections at short intervals. The number of injections and the concentration of the aripiprazole vary in view of the total amount of aripiprazole to be administered and the concentration of aripiprazole contained in each of the injectable preparations, as described above.

In some aspects, the present disclosure is directed to a prefilled syringe formulation for use in administering to a subject the above preparation, i.e., the injectable preparation, with a dosage volume of about 2.0 mL or more, for example ranging from 2.0 mL to 4.0 mL, further for example ranging from 2.2 mL to 3.5 mL, still further for example ranging from 2.3 mL to 3.4 mL, still further for example ranging from 3.0 mL to 3.2 mL, still further for example about 3.2 mL.

In some aspects, the administration of the aripiprazole injectable preparation to the subject is injected intramuscularly. In some embodiments, administering intramuscularly is at a site chosen from a deltoid, a gluteal muscle, and combinations thereof. In some embodiments, the site is the gluteal muscle. For example, depending on the number of injections for administration, the injection site may encompass various locations of the deltoid and/or gluteal muscles.

In some aspects, when the injectable preparation of the present disclosure is administered intramuscularly at a site such as a deltoid and a gluteal muscle, it is desirable that the administration may be conducted at a constant rate and at a slow speed. In some embodiments, the injectable preparation of the present disclosure is administered by slowly injecting substantially the entire contents of aripiprazole comprised in a pre-filled syringe. The term “slow” or “slowly” herein means administration over a time, e.g., about 0.5 seconds or more, about 1 second or more, about 2 seconds or more, about 3 seconds or more, about 4 seconds or more, about 5 seconds or more, about 10 seconds or more, about 15 seconds or more, about 20 seconds or more, or about 30 seconds or more, and within about 1 minute, within about 2 minutes, within about 3 minutes, within about 4 minutes, or within about 5 minutes, compared to time spent in a conventional manner in the art and/or gradual administration, and injection in a manner other than rapid and continuous manner. Administration of the preparation at a rapid speed is not desirable in terms of irritation to the injection site.

In some aspects, the methods of administering the injectable preparation of the present disclosure occur at a frequency of about once every two months. For example, the injectable preparation is administered about once every 42 to 70 days or at any integer in between and including the end points, e.g., every 49 to 63 days, e.g., every 50 to 60 days, i.e., 50, 51, 52, 53, 54, 55, 56, 57, 58, 59 or 60 days. In further aspects, the injectable preparation is administered about once every 56 days, e.g., once every 54 days to 58 days. In further aspects, the injectable preparation is administered about once every 8 weeks.

In some aspects, the injectable preparation of the present disclosure can be filled as is into a syringe for use as a prefilled syringe. This simplifies the structure of the syringe and reduces size and weight. When the injectable preparation of the present disclosure is filled into a syringe, in a further embodiment, the preparation can be administered by conducting the dispersion procedure of the present disclosure, followed by simply pressing the plunger rod of the syringe and ejecting the injectable preparation of the present disclosure through a syringe needle. This provides a prefilled syringe that offers clinical convenience and operability, thus is highly useful medically and in-dustrially. An example of producing such a prefilled syringe is such that an injectable preparation is produced in the manner as described above, the preparation is prefilled into a syringe, and then left to stand in the manner as described above to cause the injectable preparation to gel. In some aspects, the gel injectable preparation may be subjected to the dispersion procedure of the present disclosure before administration to form a homogeneous sol or suspension having a sufficient fluidity. The term “having a sufficient fluidity” in the present disclosure means, for example, that the force to eject the preparation from a syringe container comprising the preparation is 30N or less, such as, 25N or less, which may vary depending on the content of the container and/or the size of a syringe needle used. In some aspects, a syringe needle of the injectable preparation of the present disclosure is a hypodermic needle of 1 to 2 inches (i.e., 25 to 51 mm) and 21 to 23 gauges. In some aspects, a syringe needle of the injectable preparation of the present disclosure is a 1-inch (25-mm) 23 gauge needle; a 1.5-inch (38-mm) 22 gauge needle; or a 2-inch (51-mm) 21 gauge needle, and may be optionally chosen depending on the age, gender, and body type of a subject or patient. The present disclosure also includes a kit equipped with the above-described prefilled syringe.

The term “force to eject” in the present disclosure refers to the force required to eject a substantially total amount of the injectable preparation of the present disclosure from a container comprising the preparation by a rod-shaped object such as a plunger rod and corresponds to the “plunger glide force” described in the US Pharmacopeia (USP) 1382 and 382, which may be measured as sliding resistance with a measuring instrument commonly used in the art. Such a measuring instrument includes, for example, SV-52NA type tension and compression testing machine (manufactured by IMADA-SS Corporation). The term “substantially total amount” herein refers to the total content of the preparation comprised in the container before administration, or the amount after deduction of the amount remained in the container after ejection of the preparation, e.g., about 0.1 mL, about 0.2 mL, about 0.3 mL, about 0.4 mL or about 0.5 mL, from the total content.

Some embodiments are illustrated as follows.

Clause 1: A method of enhancing slidability of a container of an aripiprazole, or salt thereof, long-acting injectable preparation comprising:
dispersing a gel of the preparation to form a sol or a suspension of the preparation in the container before administration,
wherein long-acting is about two months.

Clause 2: The method according to clause 1, wherein the dispersing step comprises tapping the container at least 8 times and shaking the container for at least 8 seconds.

Clause 3: The method according to clause 1, wherein the dispersing step comprises tapping the container at least 10 times and shaking the container for at least 10 seconds.

Clause 4: The method according to any one of clauses 1 to 3, wherein a sliding resistance of the container after the dispersing step is 30N or less.

Clause 5: The method according to any one of clauses 1 to 4, wherein the container is a prefilled syringe.

Clause 6: The method according to any one of clauses 1 to 5, wherein the preparation is for intramuscular administration and for treating a subject having schizophrenia or bipolar I disorder.

Clause 7: The method according to any one of clauses 1 to 6, wherein the preparation comprises aripiprazole in an amount ranging from about 700 mg to about 1000 mg.

Clause 8: The method according to clause 7, wherein the container comprises an amount of 2.0 mL or more of the preparation per single administration.

Clause 9: The method according to any one of clauses 1 to 8, wherein the preparation is white to off-white and comprises:
720 mg or 960 mg of anhydrous aripiprazole,
carboxymethylcellulose sodium,
polyethylene glycol 400,
sodium chloride,
sodium phosphate monobasic monohydrate, and
sodium hydroxide.

Clause 10: The method according to any one of clauses 1 to 9, further comprising after dispersing, administering the preparation in the container to a subject in need thereof.

Clause 11: A method for treating a subject having schizophrenia or bipolar I disorder, comprising slowly administering the injectable preparation subjected to the method according to any one of clauses 1 to 10 to the subject, wherein slowly is over about 0.5 seconds or more.

Clause 12: A method for treating a subject having schizophrenia or bipolar I disorder, comprising dispersing an injectable preparation in a form of a gel to a sol or a suspension of aripiprazole or a salt thereof in a container, and intramuscularly administering to a subject the injectable preparation, wherein the subject is administered the injectable preparation about once every two months.

Clause 13: The method according to clause 12, wherein the dispersing step comprises tapping the container at least 8 times and shaking the container for at least 8 seconds.

Clause 14: The method according to clause 12 or 13, wherein a sliding resistance of the container after the dispersing step is 30N or less.

Clause 15: The method according to any one of clauses 12 to 14, wherein the container is a prefilled syringe.

Clause 16: The method according to any one of clauses 12 to 15, wherein the administration is at a gluteal muscle of the subject.

Clause 17: The method according to any one of clauses 12 to 16, wherein the administration of about once every two months results in a total amount of aripiprazole ranging from about 700 mg to about 1000 mg to the subject.

Clause 18: The method according to any one of clauses 12 to 17, wherein the container comprises an amount of 2.0 mL or more of the preparation per single administration.

Clause 19: The method according to any one of clauses 12 to 18, wherein the preparation is white to off-white and comprises:
720 mg or 960 mg of anhydrous aripiprazole,
carboxymethylcellulose sodium,
polyethylene glycol 400,
sodium chloride,
sodium phosphate monobasic monohydrate, and
sodium hydroxide.

Clause 20: The method according to any one of clauses 12 to 19, wherein the administration comprises slowly injecting the injectable preparation to the subject and slowly is over about 0.5 seconds or more.

Clause 21: A long-acting injectable preparation for intramuscular administration for treating a subject having schizophrenia or bipolar I disorder, comprising aripiprazole or a salt thereof in a container, wherein the preparation is in a form of a gel upon static conditions and reverts to a sol or a suspension when dispersed and long-acting is about two months.

Clause 22: The preparation according to clause 21, wherein the dispersion comprises tapping the container at least 8 times and shaking the container for at least 8 seconds.

Clause 23: The preparation according to clause 21 or 22, wherein a sliding resistance of the container after the preparation is dispersed is 30N or less.

Clause 24: The preparation according to any one of clauses 21 to 23, wherein the preparation before the dispersion is in the form of a gel and the preparation after the dispersion is in the form of a sol.

Clause 25: The preparation according to any one of clauses 21 to 24, wherein the administration is injection at a gluteal muscle of the subject.

Clause 26: The preparation according to any one of clauses 21 to 25, wherein the preparation comprises a total amount of aripiprazole ranging from about 700 mg to about 1000 mg.

Clause 27: The preparation according to any one of clauses 21 to 26, wherein the preparation is a prefilled syringe for the administration in an amount of 2.0 mL or more per single administration.

Clause 28: The preparation according to any one of clauses 21 to 27, wherein the preparation is white to off-white and comprises:
720 mg or 960 mg of anhydrous aripiprazole,
carboxymethylcellulose sodium,
polyethylene glycol 400,
sodium chloride,
sodium phosphate monobasic monohydrate, and
sodium hydroxide.

Clause 29: Use of aripiprazole or a salt thereof in the manufacture of a medicament for treating a subject having schizophrenia or bipolar I disorder, wherein the medicament is a long-acting injectable preparation for intramuscular administration comprising aripiprazole or a salt thereof in a container, the preparation is in a form of a gel upon static conditions and reverts to a sol or a suspension when dispersed, and long-acting is about two months.

Clause 30: The use according to clause 29, wherein the dispersion comprises tapping the container at least 8 times and shaking the container for at least 8 seconds.

Clause 31: The use according to clause 29 or 30, wherein a sliding resistance of the container after the preparation is dispersed is 30N or less.

Clause 32: The use according to any one of clauses 29 to 31, wherein the preparation before the dispersion is in the form of a gel and the preparation after the dispersion is in the form of a sol.

Clause 33: The use according to any one of clauses 29 to 32, wherein the administration is injection at a gluteal muscle of the subject.

Clause 34: The use according to any one of clauses 29 to 33, wherein the preparation comprises a total amount of aripiprazole ranging from about 700 mg to about 1000 mg.

Clause 35: The use according to any one of clauses 29 to 34, wherein the preparation is a prefilled syringe for the administration in an amount of 2.0 mL or more per single administration.

Clause 36: The use according to any one of clauses 29 to 35, wherein the preparation is white to off-white and comprises:
720 mg or 960 mg of anhydrous aripiprazole,
carboxymethylcellulose sodium,
polyethylene glycol 400,
sodium chloride,
sodium phosphate monobasic monohydrate, and
sodium hydroxide.

Clause 37: The use according to any one of clauses 29 to 36, wherein the administration comprises slowly injecting the injectable preparation to the subject and slowly is over about 0.5 seconds or more.

Clause 38: A method of enhancing slidability of a container of a long-acting intramuscular injectable preparation comprising aripiprazole or a salt thereof, comprising:
consecutively tapping and shaking the container before administration in a form of a gel to a sol or a suspension of the preparation,
wherein the container comprises a dosage volume of 2.0 mL or more of the preparation per dose,
a sliding resistance of the container after the tapping and shaking is 30N or less, and long-acting is about two months.

Clause 39: The method according to clause 38, wherein the administration comprises slowly injecting the injectable preparation to the subject and slowly is over about 0.5 seconds or more.

Clause 40: The method according to clause 38 or 39, wherein the preparation comprises aripiprazole in an amount ranging from about 700 mg to about 1000 mg.

In one illustrative embodiment, an injectable preparation of the present disclosure is a white to off-white, aqueous extended-release suspension for intramuscular injection in the form of a pre-filled syringe, comprising:
a strength of 720 mg or 960 mg anhydrous aripiprazole,
carboxymethylcellulose sodium (5 mg),
polyethylene glycol 400 (1 mg),
sodium chloride (6.1 mg),
sodium phosphate monobasic monohydrate (0.74 mg), and
sodium hydroxide to adjust pH.

### EXAMPLES

The present invention is illustrated in more detail with examples and test examples below, but is not intended to be limited thereto.

### Example 1: Vibration Procedure to Aripiprazole Injectable Preparation

### (Transportation Simulation)

The aripiprazole injectable preparation prepared according to the method described in PTL 2 was subjected to a vibration procedure empirically reproducing transportation environments as a transportation simulation, followed by dispersion procedures under various conditions, and the redispersion condition to enhance slidability of the injectable preparation was determined.

(1) An Injectable Preparation Sample Used in this Example

The preparation of Table 1 was loaded 3.3 mL into a 5-mL TopPac® (brand name) syringe of Schott as a sample used for this example, and a vibration test was conducted for this sample under reduced pressure. The syringe is a plastic syringe consisting of cyclic olefin copolymers (COC), which meets the standard of the Container Closure Integrity (CCI) at −80° C.

TABLE 1
Preparation used

| Composition (mg/mL) | | |
|---|---|---|
| Aripiprazole | 300 | mg |
| Sodium carboxymethyl cellulose | 5 | mg |
| Povidone K17 | 4 | mg |
| Polyethylene glycol 400 | 1 | mg |
| Sodium chloride | 6.1 | mg |
| Sodium hydrogen phosphate | 0.74 | mg |
| monohydrate | | |

| | |
|---|---|
| Sodium hydroxide | Q.S. to pH 7 |
| Water for injection | Q.S. to 1 mL |

### (2) Vibration Test (Transportation and Conveyance)

A vibration test was conducted in a manner according to ASTM D4169-16 of the American Society for Testing and Material (ASTM). Specifically, vibration tests were conducted with an electrodynamic vibration test system i240/SA3M vertical composite type manufactured by IMV Corporation at 20° C. to 25° C. under reduced pressure under the conditions of Tables 2 to 4.

TABLE 2
ASTM D 4169-16 Truck conditions Medium level

| Frequency | PSD | | | Environment |
|---|---|---|---|---|
| (Hz) | (G²/Hz) | Time | Direction | temperature |
| 3 | 0.018 | 3 hours | Up and down | 23 ± 2° C. |
| 4 | 0.018 | | | |
| 6 | 0.00072 | | | |
| 12 | 0.00072 | | | |
| 16 | 0.0036 | | | |
| 25 | 0.0036 | | | |
| 30 | 0.00072 | | | |
| 40 | 0.0036 | | | |
| 80 | 0.0036 | | | |
| 100 | 0.00036 | | | |
| 200 | 0.000018 | | | |

PSD = Power Spectral Density

TABLE 3
ASTM D 4169-16 Air condition level 2

| Frequency | PSD | | | Environment | Environment |
|---|---|---|---|---|---|
| (Hz) | (G²/Hz) | Time | Direction | temperature | pressure |
| 2 | 0.0002 | 1 hour | Up and | 23 ± 2° C. | 0.7 ± 0.05 |
| 12 | 0.01 | | down | | atm |
| 100 | 0.01 | | | | |
| 300 | 0.00001 | | | | |

PSD = Power Spectral Density

TABLE 4
ASTM D 4169-16 Air condition level 2

| | Action | Number | | Environment | Wave |
|---|---|---|---|---|---|
| Acceleration | time | of impact | Direction | temperature | shape |
| 20 G | 15 ms | 20 times | Up | 23 ± 2° C. | Half sine |
| | | | | | curve |

### Example 2: Dispersion Test

The changes of slidability after dispersion procedures in samples before and after the vibration test of Example 1 are shown in Tables 5 and 6. A syringe comprising the injectable preparation was subjected to dispersion procedures by hand with certain times of tapping and shaking, and then the syringe was connected to a 22G 1.5-inch injection needle (manufactured by Terumo Corporation). The slidability of the syringe was measured with SV-52NA type tension and compression testing machine (manufactured by IMADA-SS Corporation) under the conditions of Range: A (50N), Displacement polarity: DOWN, Load polarity mode: PUSH, Test rate: 100 mm/min (V2). The slidability was measured based on the force (sliding resistance) to eject the preparation in the syringe with a plunger rod, i.e., a plunger glide force in accordance with the US Pharmacopeia (USP).

TABLE 5
Samples before vibration test

| | |
|---|---|
| Maximum Glide Force | Shaking (Seconds) |

| | | | | |
|---|---|---|---|---|
| (N) | 0 | 5 | 10 | 15 |

| | | | |
|---|---|---|---|
| Tapping | 0 | 22.9 | |
| (Times) | 5 | | |
| | 10 | | 21.2 |
| | 15 | | |

TABLE 6
Samples after vibration test

| | |
|---|---|
| Maximum Glide Force | Shaking (Seconds) |

| | | | | |
|---|---|---|---|---|
| (N) | 0 | 5 | 10 | 15 |

| | | | | | |
|---|---|---|---|---|---|
| Tapping | 0 | 48.4 | 47.9 | 45.4 | 43.2 |
| (Times) | 5 | 48.4 | 35.5 | | |
| | 10 | 45.9 | | 20.0 | |
| | 15 | 31.4 | | | 19.6 |

### Example 3: Measurement of Slidability Signals

The slidability signals of several samples were measured. The measuring instrument and conditions were the same as those in Example 2.

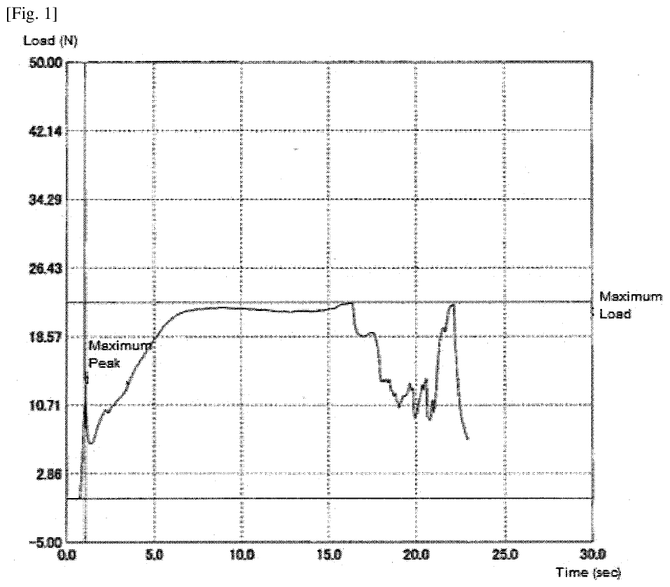

The slidability signals of a sample before the vibration procedure (transportation simulation) are shown in FIG. 1.

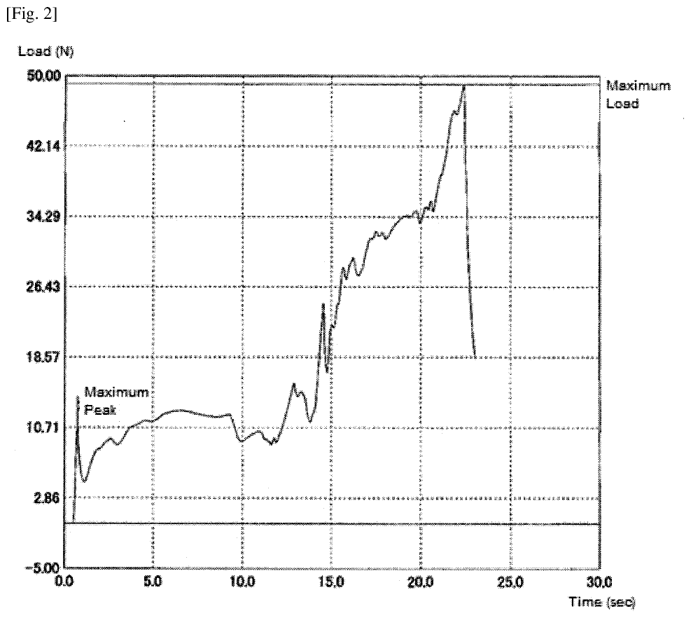

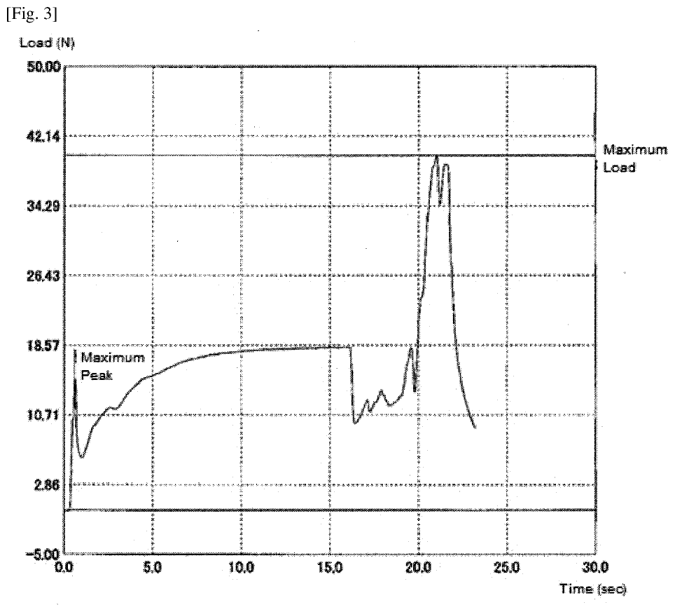

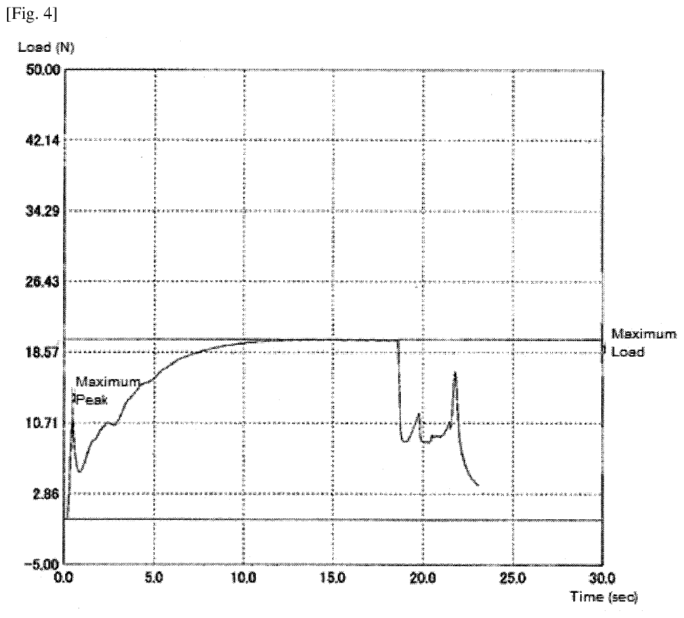

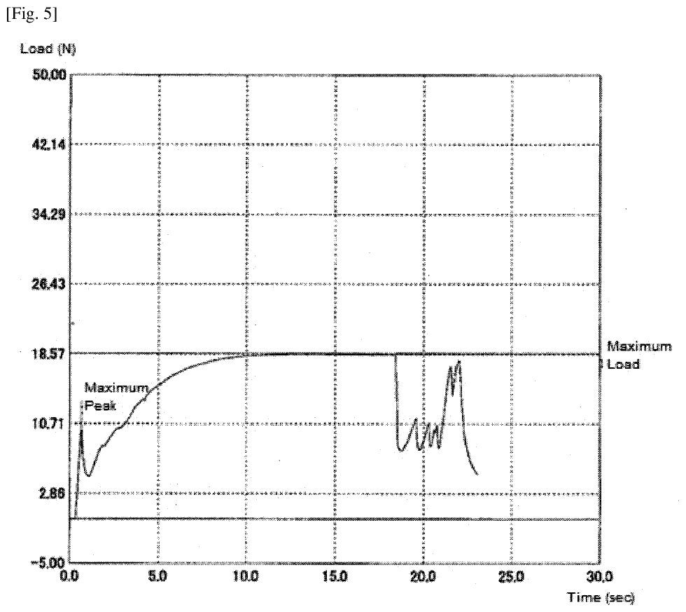

The slidability signals of samples conducted dispersion procedures of 0 times of tapping and 0 seconds of shaking, 5 times of tapping and 5 seconds of shaking, 10 times of tapping and 10 seconds of shaking, or 15 times of tapping and 15 seconds of shaking after the transportation simulation are shown in FIGS. 2, 3, 4, and 5, respectively.

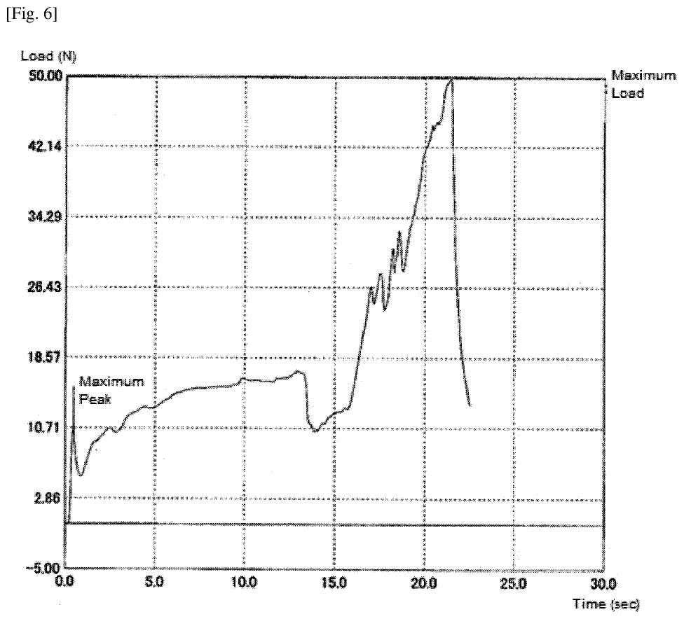

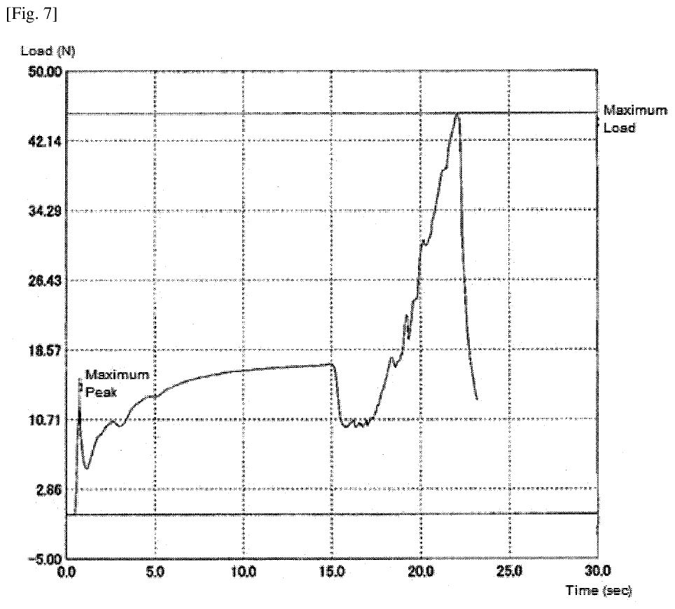

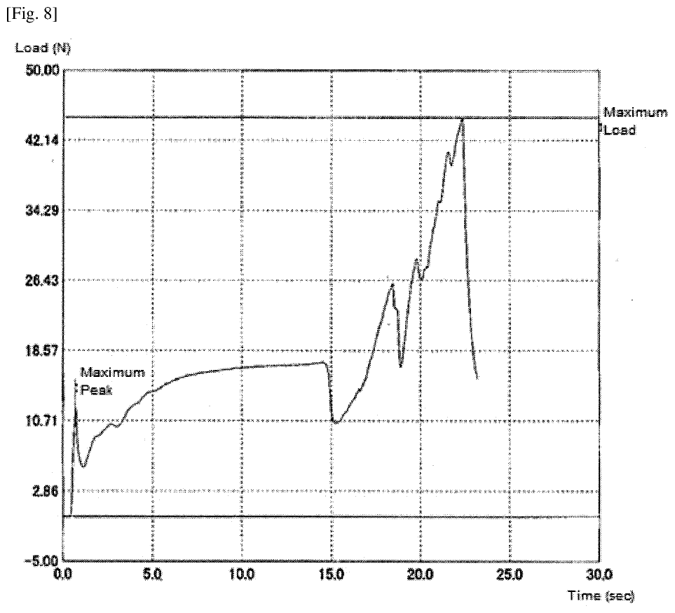

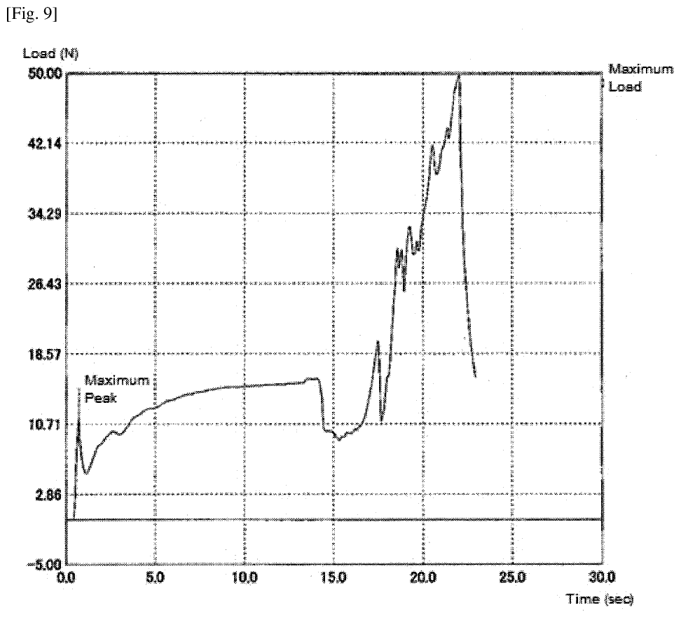

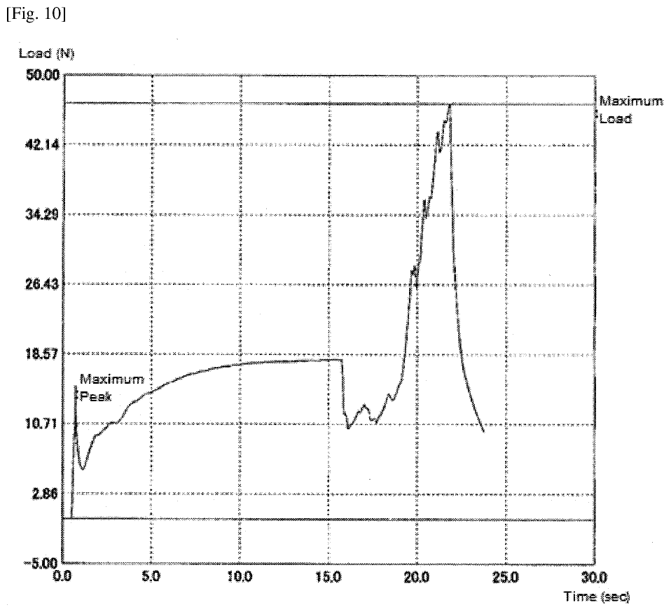

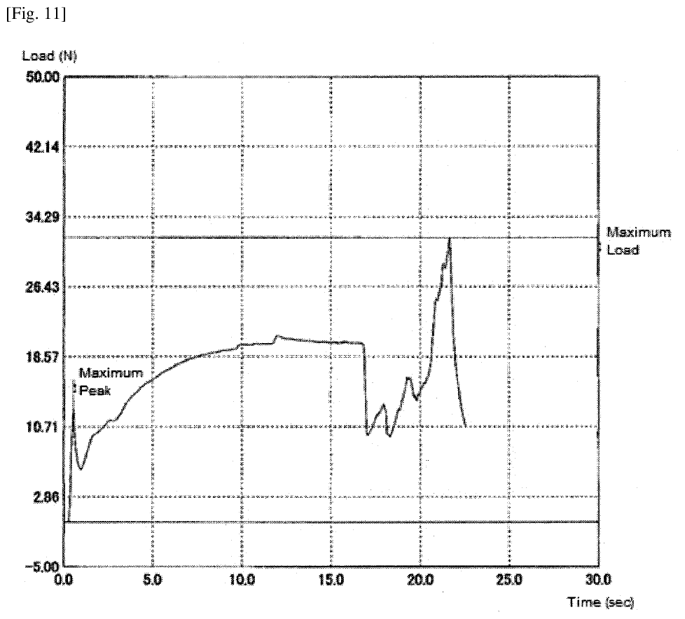

The slidability signals of samples conducted dispersion procedures of 5 times of tapping and 0 seconds of shaking, 10 times of tapping and 0 seconds of shaking, 15 times of tapping and 0 seconds of shaking, 0 times of tapping and 5 seconds, 0 times of tapping and 10 seconds, or 0 times of tapping and 15 seconds of shaking after the transportation simulation are shown in FIGS. 6, 7, 8, 9, 10, and 11, respectively.

For samples after the transportation simulation, the samples conducted dispersion procedures of 10 times of tapping and 10 seconds of shaking, and 15 times of tapping and 15 seconds of shaking showed a maximum glide force of 30N or less.

### Example 4: Transportation Test

In order to confirm changes of slidability after actual transportation of samples, the preparation same as Example 1 was stored into a 10-mL vial and was let make a round trip between Japan and the United States by air (stored at 1° C. to 15° C.). Then, the change of slidability was determined. The results showed that after the round trip, a part of the samples may result in insufficient contents of the preparation to be ejected without sufficient tapping and shaking (dispersion procedures).

All publications and patents mentioned herein are hereby incorporated by reference in their entirety as if each individual publication or patent was specifically and indi-vidually indicated to be incorporated by reference.

Claims or descriptions that include “or” or “and/or” between at least one members of a group are considered satisfied if one, more than one, or all of the group members are present in, employed in, or otherwise relevant to a given product or process unless indicated to the contrary or otherwise evident from the context. The disclosure includes embodiments in which exactly one member of the group is present in, employed in, or otherwise relevant to a given product or process. The disclosure includes embodiments in which more than one, or all the group members are present in, employed in, or otherwise relevant to a given product or process.

Furthermore, the disclosure encompasses all variations, combinations, and per-mutations in which at least one limitation, element, clause, and descriptive term from at least one of the listed claims is introduced into another claim. For example, any claim that is dependent on another claim can be modified to include at least one limitation found in any other claim that is dependent on the same base claim. Where elements are presented as lists, e.g., in Markush group format, each subgroup of the elements is also disclosed, and any element(s) can be removed from the group. It should be understood that, in general, where the disclosure, or aspects of the disclosure, is/are referred to as comprising particular elements and/or features, embodiments of the disclosure or aspects of the disclosure consist, or consist essentially of, such elements and/or features. For purposes of simplicity, those embodiments have not been specifically set forth in haec verba herein. Where ranges are given, endpoints are included. Furthermore, unless otherwise indicated or otherwise evident from the context and understanding of one of ordinary skill in the art, values that are expressed as ranges can assume any specific value or sub range within the stated ranges in different embodiments of the disclosure, to the tenth of the unit of the lower limit of the range, unless the context clearly dictates otherwise.

Those of ordinary skill in the art will recognize or be able to ascertain using no more than routine experimentation, many equivalents to the specific embodiments of the disclosure described herein. Such equivalents are intended to be encompassed by the following claims.

### INDUSTRIAL APPLICABILITY

The dispersion procedure described herein allows for more stable provision of aripiprazole injectable preparations.

1. A method of enhancing slidability of a container of an aripiprazole, or salt thereof, long-acting injectable preparation comprising:
dispersing a gel of the preparation to form a sol or a suspension of the preparation in the container before administration,
wherein long-acting is about two months.

2. The method according to claim 1, wherein the dispersing step comprises tapping the container at least 8 times and shaking the container for at least 8 seconds.

3. The method according to claim 1, wherein the dispersing step comprises tapping the container at least 10 times and shaking the container for at least 10 seconds.

4. The method according to claim 1, wherein a sliding resistance of the container after the dispersing step is 30N or less.

5. The method according to claim 1, wherein the container is a prefilled syringe.

6. The method according to claim 1, wherein the preparation is for intramuscular administration and for treating a subject having schizophrenia or bipolar I disorder.

7. The method according to claim 1, wherein the preparation comprises aripiprazole in an amount ranging from about 700 mg to about 1000 mg.

8. The method according to claim 7, wherein the container comprises an amount of 2.0 mL or more of the preparation per single administration.

9. The method according to claim 1, wherein the preparation is white to off-white and comprises:
720 mg or 960 mg of anhydrous aripiprazole,
carboxymethylcellulose sodium,
polyethylene glycol 400,
sodium chloride,
sodium phosphate monobasic monohydrate, and
sodium hydroxide.

10. The method according to claim 1, further comprising after dispersing, administering the preparation in the container to a subject in need thereof.

11. A method for treating a subject having schizophrenia or bipolar I disorder, comprising slowly administering the injectable preparation subjected to the method according to claim 1 to the subject, wherein slowly is over about 0.5 seconds or more.

12. A method for treating a subject having schizophrenia or bipolar I disorder, comprising dispersing an injectable preparation in a form of a gel to a sol or a suspension of aripiprazole or a salt thereof in a container, and intramuscularly administering to a subject the injectable preparation, wherein the subject is administered the injectable preparation about once every two months.

13. The method according to claim 12, wherein the dispersing step comprises tapping the container at least 8 times and shaking the container for at least 8 seconds.

14. The method according to claim 12, wherein a sliding resistance of the container after the dispersing step is 30N or less.

15. The method according to claim 12, wherein the container is a prefilled syringe.

16. The method according to claim 12, wherein the administration is at a gluteal muscle of the subject.

17. The method according to claim 12, wherein the administration of about once every two months results in a total amount of aripiprazole ranging from about 700 mg to about 1000 mg to the subject.

18. The method according to claim 12, wherein the container comprises an amount of 2.0 mL or more of the preparation per single administration.

19. The method according to claim 12, wherein the preparation is white to off-white and comprises:
720 mg or 960 mg of anhydrous aripiprazole,
carboxymethylcellulose sodium,
polyethylene glycol 400,
sodium chloride,
sodium phosphate monobasic monohydrate, and
sodium hydroxide.

20. The method according to claim 12, wherein the administration comprises slowly injecting the injectable preparation to the subject and slowly is over about 0.5 seconds or more.

21. A long-acting injectable preparation for intramuscular administration for treating a subject having schizophrenia or bipolar I disorder, comprising aripiprazole or a salt thereof in a container, wherein the preparation is in a form of a gel upon static conditions and reverts to a sol or a suspension when dispersed and long-acting is about two months.

22. The preparation according to claim 21, wherein the preparation comprises a total amount of aripiprazole ranging from about 700 mg to about 1000 mg.

23. The preparation according to claim 21, wherein the preparation is a prefilled syringe for the administration in an amount of 2.0 mL or more per single administration.

24. The preparation according to claim 21, wherein the preparation is white to off-white and comprises:
720 mg or 960 mg of anhydrous aripiprazole,
carboxymethylcellulose sodium,
polyethylene glycol 400,
sodium chloride,
sodium phosphate monobasic monohydrate, and
sodium hydroxide.
